(19) United States
(12) Patent Application Publication
(10) Pub. No.: US 2017/0000346 A1
(43) Pub. Date: Jan. 5, 2017

(54) Wireless Charging And Pairing Of Wireless Associated Devices

(71) Applicant: General Electric Company, Schenectady, NY (US)

(72) Inventor: David Wayne Duckert, Wauwatosa, WI (US)

(21) Appl. No.: 14/755,243

(22) Filed: Jun. 30, 2015

(51) Int. Cl. A61B 5/00; H02J 7/04; A61B 5/0402; H02J 7/02

(52) U.S. Cl. CPC A61B 5/0006; H02J 7/025; H02J 7/04; A61B 5/0402; A61B 2560/0214

(57) ABSTRACT

An ECG monitoring device includes an acquisition module that is wirelessly connected to a host module. The acquisition module includes an inductive charging/receiving coil connected to a re-chargeable battery within the acquisition module. The host module includes an inductive charging coil operably connected to a power source for the host module. When the acquisition module is connected to the host module, the charging/receiving coil is positioned in alignment with the inductive charging coil to enable the battery to be charged via the signals emitted from the inductive charging coil and received by the charging/receiving coil. The receiving coil in the acquisition module also sends data to the host module while the acquisition module is positioned within the holster. This data connection is used to send different types of data, and particularly to send module pairing or identifier data utilized to pair or associate the acquisition module with the host module.

### BACKGROUND OF THE INVENTION

The invention relates generally to monitoring devices and equipment for obtaining and illustrating data about a patient to which the equipment is connected, and more particularly to monitoring devices and methods for connecting or pairing wireless data or signal acquisition devices with their associated host monitoring devices.

In monitoring or diagnostic devices that are currently utilized, high performance electrocardiographs (ECG) often have two distinct components, the acquisition module and the host module. In operation, the acquisition module detects the analog ECG signals from the patient through sensors attached to the patient and connected to the acquisition module, and converts these signals into a digital signal that is transmitted to the host module. The host module receives the digitally encoded data signals from the acquisition module and performs various functions with the digital data signals, such as by presenting the data on the screen of the host module, providing arrhythmia analysis, creating and storing reports, and interfacing with other hospital systems to provide the data to those systems, among others.

In many prior art monitoring devices 100, such as shown in FIG. 1, the sensors 101 attached to the patient 102 are connected to the acquisition module 104 by wires or leads 106, and the acquisition module 104 is connected directly to the host module 108 by a wired connection or cable 110. The acquisition module 104 is normally carried on the host module 108 by means of a holster 112 in which the acquisition module 104 can be releasably positioned. In use, the acquisition module 104 can be removed from the holster 112 when the sensors 101 are applied to the patient 102 and is returned to the holster 112 on the host module 108 when the testing is completed.

Because ECG signal levels are very small, the best quality signals are captured by keeping the acquisition module 104 as close as possible to the patient 102. As a result, the leads 106 short in order to limit the introduction of noise resulting from long leads. However, with short leads 106, the placement of the acquisition module 104 and host module 108 relative to one another and to the patient 102 is limited.

As an alternative to the prior art ECG devices 100 in FIG. 1, other prior art ECG devices 200, such as shown in FIG. 2, have been developed that replace the wired connection 110 in the prior device 100 with a wireless connection or link 210 between the acquisition module 204 and the host module 208. This device 200 effectively frees the acquisition module 204 from the physical limitation of the lead 110 between the host module 208. As a result, the patient 202 to which the acquisition module 204 is connected can move about more freely, thereby facilitating other testing of the patient 202, such as stress tests which require the patient 202 to walk on a treadmill. In addition, the wireless connection 210 allows the host module 208 to maintain a distance from the patient 202 sufficient that the host module 208 does not have to be cleaned/disinfected, and to reduce the number of cables (not shown) connecting the host module 208 to various items, such as a wall outlet (not shown), to reduce tripping/tangling hazards.

However, the wireless link 210 in the device 200 is not without certain shortcomings. One issue is that the acquisition module 204 needs to be associated or paired with the host module 208 in order to ensure that data obtained by sensors 201 connected to the acquisition module 204 by wires 206 is sent wirelessly from the acquisition module 204 is received by the proper or associated host module 208. Looking at FIG. 3, the acquisition modules 204,204′ of two separate devices 200,200′ operating on two patients 202,202′ in close proximity could inadvertently become paired with the wrong host module 208,208′. In this situation, ECG data from patient 202 could be wirelessly transmitted to the host module 208′ assigned to patient 202′, and vice versa.

Various attempts to prevent data mixing from occurring between devices 200,200′ have been employed, including temporarily connecting the acquisition module 204 to the host module 208 to pair the modules 204,208 in some manner, or by entering pairing codes into one or both of the acquisition module 204 and host module 208. One other attempt to solve this issue is the use of near field communication (NFC) systems to associate or pair the acquisition module 204 with the host module 208 for a wireless device 200. When using an NCF system in a wireless device 200, the acquisition device 204 and the host device 208 are brought into very close proximity, e.g., touching, and are automatically linked, such as by transmitting a digital code from the host module 208 to the acquisition modules 204 that is incorporated into all signals sent from the acquisition module 204 to the host module 208. If the code from the acquisition module 204 matches the code stored by the host module 208, the host module 208 recognizes the signal as coming from the proper, paired acquisition module 204 and further processes the signal. If the codes do not match, the host module 208 simply ignores the incoming digital data signal.

While NFC systems and other modes for entering association codes in the acquisition modules 204,204′ and or host modules 208,208′ can reduce data mixing between devices 200,200′, the connection or pairing between the modules 204,208 does require an additional step, and thus additional time, as opposed to prior wired devices 100. Further, the pairing method must often be repeated in order to confirm that the acquisition module 204 is associated with the particular host module 208 to avoid data mixing, thereby requiring still further time for completion.

Further, another issue with wireless devices 200 is that due to its disconnected state, the acquisition module 204 must contain a battery, (not shown) which periodically needs to be re-charged for proper operation of the acquisition module 204. A number of different conventional charging structures and methods are available to re-charge the battery within the acquisition module 204, such as a docking station positioned within the holster, but connector alignment and exposed connector pins are an issue.

Therefore, in order to address the issues discussed above regarding the wired and wireless versions of current ECG monitoring devices, it is desirable to develop a monitoring device and system that provides the freedom of movement associated with the wireless device, along with features designed to address the module pairing and charging issues.

### BRIEF DESCRIPTION OF THE INVENTION

In embodiments of the invention, an ECG or other type of monitoring device includes an acquisition module that is wirelessly connected to a host module. The acquisition module includes an inductive charging/receiving coil operably connected to a re-chargeable battery present within the acquisition module. The host module includes a holster with an inductive charging coil operably connected to a power source for the host module, such as an electrical wall outlet. When the acquisition module is placed in the holster, the charging/receiving coil is positioned in alignment with the inductive charging coil in the holster in order to enable the battery can be charged via the signals emitted from the inductive charging coil and received by the charging/receiving coil. In this embodiment, the charging/receiving coil in the acquisition module is disposed within the acquisition module, negating the need for exposed charging connection ports or pins.

According to another aspect of an exemplary embodiment of the invention, the inductive charging scheme used to re-charge the battery in the acquisition module also sends data between the host module and the acquisition module while the acquisition module is positioned within the holster. This data connection can be used send different types of data, and particularly to send module pairing or identifier data utilized to associate the acquisition module with the host module. As a result, each time the acquisition module is placed in the holster of the host module, the inductive charging coil in the holster begins charging the acquisition module and concurrently re-associates or pairs the acquisition module with the host module via the inductive charging data link, eliminating the need for NFC or other association methods.

According to another aspect of an exemplary embodiment of the invention, a medical monitoring device includes an acquisition module configured to receive incoming data signals from a number of sensors connected to the acquisition module concerning a physiological parameter, a host module configured to receive wireless signals from the acquisition module regarding the incoming data signals received by the acquisition module and a module pairing and charging system selectively connected between the acquisition module and the host module and configured to pair the acquisition module with the host module while charging the acquisition module.

According to still another aspect of an exemplary embodiment of the invention, a method for pairing an acquisition module of a medical monitoring device with a host module of the medical monitoring device includes the steps of providing a first portion of a pairing and charging system on the host module, providing a second portion of the pairing and charging system on the acquisition module, aligning the second portion with the first portion, transmitting power from the first portion to the second portion and transmitting a unique pairing identifier from the second portion to the first portion.

According to still a further aspect of an exemplary embodiment of the invention, a medical monitoring device for providing information about patient operably connected to the device includes an acquisition module including a number of sensors adapted to be attached to the patient to detect a physiological parameter of the patient and a wireless transmitter to send data signals representative of the sensed physiological parameter a host module including a wireless receiver to receive the wireless data signals from the acquisition module and a display to visually represent the data signals and an inductive module pairing and charging system selectively connected between the acquisition module and the host module. The module pairing and charging system includes a first portion disposed on the host module and including a driver operably connected to a power source and a transmitting coil connected to the driver and a second portion disposed on the acquisition module and including a unique device identifier stored in an electronic storage medium, a data transmitter operably connected to the electronic storage medium and a receiving coil operably connected to the data transmitter.

It is to be understood that both the foregoing general description and the following detailed description are exemplary and explanatory and are intended to provide further explanation of the invention as claimed.

### BRIEF DESCRIPTION OF THE DRAWINGS

FIG. 1 is a schematic view of a prior art ECG monitoring device using a wired connection between the acquisition module and the host module.

FIG. 2 is a schematic view of a prior art ECG monitoring device using a wireless connection between the acquisition module and the host module.

FIG. 3 is a schematic view of prior art ECG monitoring devices using a wireless connection in a data mixing situation.

FIG. 4 is a schematic view of a prior art ECG monitoring device using a wireless connection between the acquisition module and the host module and employing an NFC pairing mode.

FIG. 5 an isometric view of an ECG monitoring device using a wireless charging and data connection between the acquisition module and a holster of the host module of the monitoring device in accordance with an exemplary embodiment of the invention.

FIG. 6 is a cross-sectional view along line 6-6 of FIG. 5 of the acquisition module and holster in accordance with an exemplary embodiment of the invention.

FIG. 7 is a schematic view of the inductive power and data transmission system of the ECG monitoring device in accordance with an exemplary embodiment of the invention.

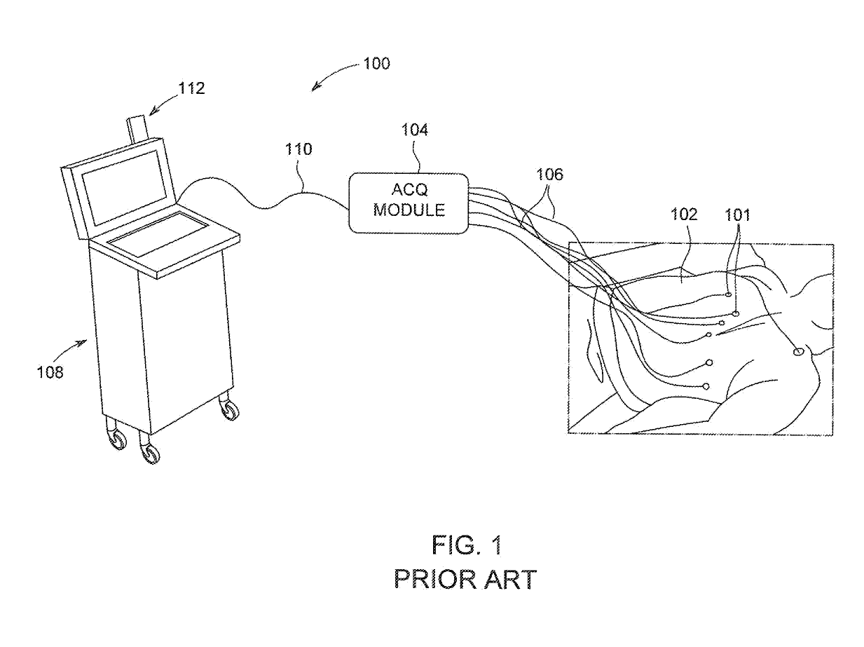

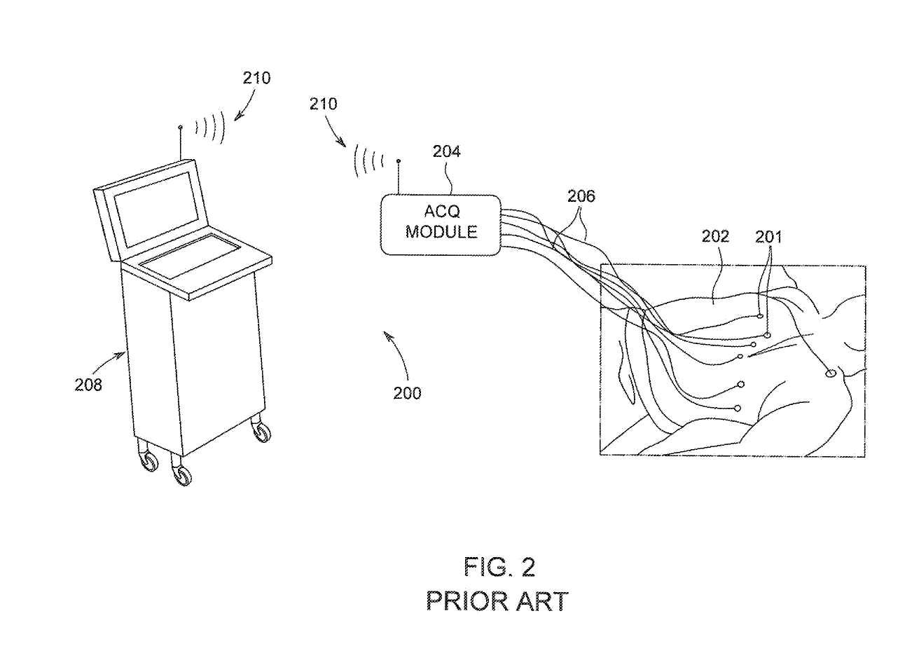

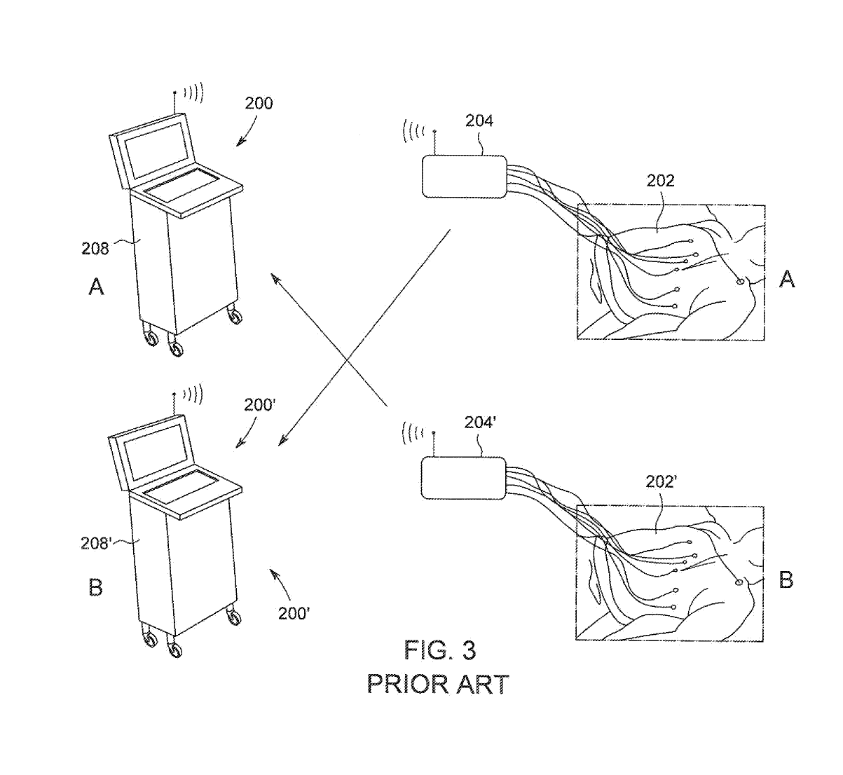

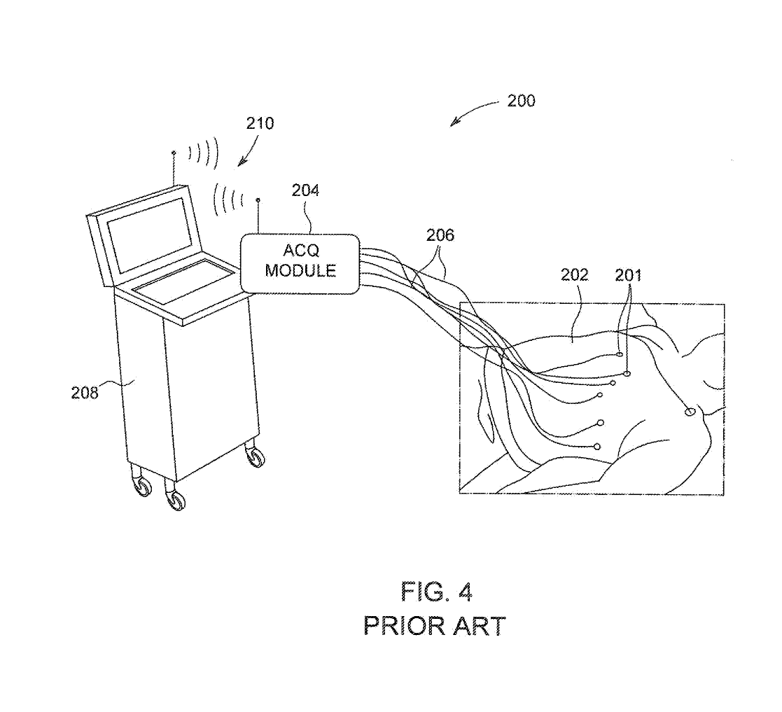

### DETAILED DESCRIPTION OF THE INVENTION

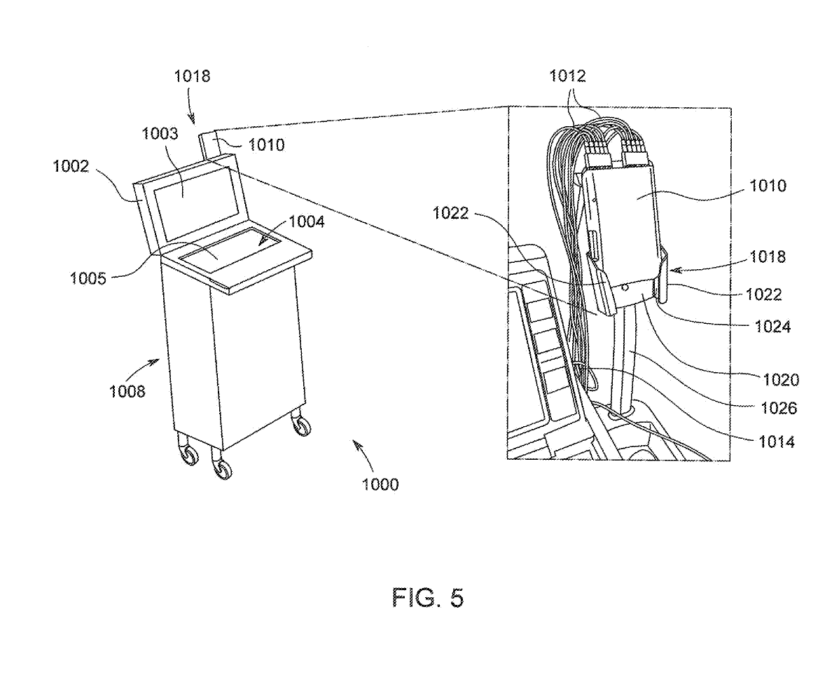

FIG. 5 illustrates an exemplary embodiment of the invention which includes a monitoring device 1000, which can be any suitable type of monitoring device for monitoring various parameters of a patient (not shown) operably connected to the device 1000, such as an ECG monitoring device. The device 1000 includes a display 1002 of any suitable type, such as a touch screen display, having a screen 1003 thereon on which the monitoring data signals (not shown) regarding the patient connected to the device 1000 can be displayed or otherwise visually represented. When formed as a touch screen, the display 1002 can additionally function as a user interface 1004 for use in controlling the operation of the device 1000, though the interface 1004 can be formed as a separate component connected to the device 1000, such as a keyboard 1005, mouse or touch pad, among others, if desired.

In the exemplary embodiment of FIG. 5, the device 1000 takes the form of a medical monitoring device that includes a host module 1008, to which the display 1002 and interface 1005 are connected, and an acquisition module 1010.

The host module 1008 includes a central processing unit (CPU) (not shown) disposed within the host module 1008 and operable to receive and process data from the acquisition module 1010 on the ECG parameters of the patient, though monitoring by the device 1000 other bodily functions or systems are also contemplated as being within the scope of the present invention. The host module 1008 can also include other components such as a memory module (not shown), which can take the form of any suitable computer-readable storage media, for example a RAM module, and an electronic storage medium, component or database (not shown), each of which are operably connected to the CPU in order to assist in the monitoring function of the host module 1008 using the data signals supplied to the CPU via the acquisition module 1010. The host module 1008 may also include an audio speaker (not shown) for enabling the host module 1008 to provide audible indications of the ECG and other various physiological parameters of the patient monitored by the host module 1008.

The data concerning the ECG signals can then be transmitted from the CPU to the display 1002 for presentation in a specified manner on the screen 1003 of the display 1002 for review by an individual monitoring the patient via the display 1002 and/or to a network (not shown) to which the device 1000 is operably connected.

The acquisition module 1010 has one or more leads 1012 connected in any suitable manner to the acquisition module 1012 that terminate opposite the acquisition module 1010 in ECG electrodes 1014 in order to monitor the ECG parameters of the patient. The electrodes 1014 are connected to the patient to receive and transmit the signals from the patient back to the acquisition module 1010 along their respective leads 1012. The acquisition module 1010 then wirelessly transmits the signals to the host module 1008 for processing, as discussed previously.

In order to effect the wireless transmission of the data from the acquisition module 1010 to the host module 1008, the acquisition device 1010 must include a unique identification code that is transmitted along with the data and recognized by the CPU of the host module 1008. The initial transmission of the unique identifier of the acquisition module 1010 to pair or associate the host module 1008 with the acquisition module 1010 is performed by a combined module pairing and charging system 1016.

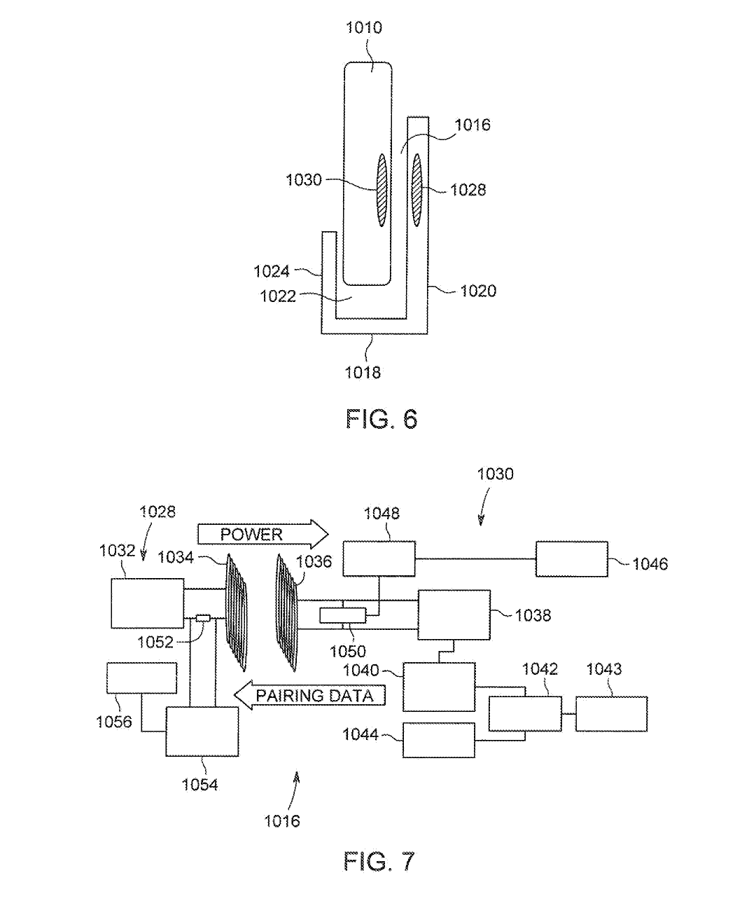

Referring to FIGS. 6 and 7, in an exemplary embodiment of the device 1000, the host module 1008 includes a holster 1018. The holster 101 8 includes a base 1020 and a pair of opposed arms 1022 that extend outwardly from the base 1020 to define a slot 1024 therebetween shaped complementary to the acquisition module 1010. The base 1020 is mounted to the host module 1008 via a suitable post 1026, such that the acquisition module 1010 can be inserted into and retained within the slot 1024 by the arms 1022. Alternatively, the holster 1022 can have any other suitable configuration capable of holding the acquisition module 1010 securely on the host module 1008, such as with more or fewer arms 1022, or with any suitable alternative securing mechanism (not shown) such as an enclosure or pocket or other engagement member, such as a strap, among others.

The module pairing and charging system 1016 is an inductive module pairing and charging system formed with a first portion 1028 disposed within the base 1020 of the holster 1018 and a second portion 1030 disposed within the acquisition module 1010. When the acquisition module 1010 is placed within the holster 1018, the second portion 1030 of the system 1016 is placed in alignment with an in close proximity to the first portion 1028 disposed in the base 1020, as best shown in the exemplary embodiment of FIG. 6.

Referring now to the illustration of the exemplary embodiment of the module pairing and charging system 1016 in FIG. 7, the first portion 1028 includes a driver 1032 that impresses an AC waveform, such as from a power supply (not shown) operably connected to the host module 1008, across a transmitting coil 1034. Energy from the waveform is transmitted from the transmitting coil 1034 to the receiver coil 1036 located in the second portion 1030 as a result of the close proximity of the transmitting coil 1034 and the receiver coil 1036 when the acquisition module 1010 is placed in the holster 1018.

From the receiving coil 1036, a rectifier 1038 converts this to a DC voltage which can be regulated by a voltage regulator 1040 operably connected thereto. Suitable power management circuitry 1042 connected to the voltage regulator 1040 is used to charge the battery 1044 as well as determine if the acquisition module 1010 is being externally powered or if it should run from the battery 1044. The power management circuitry 1042 additionally regulates the operation of the battery 1044 to supply power to all of the other circuitry 1043 within the acquisition module 1010 to provide the functionality of the acquisition module 1010.

For the purposes of associating or pairing the acquisition module 1010 with the host module 1008, the second portion 1030 of the system 1016 within the acquisition module 1010 contains a unique device identifier or ID 1046 retained in a suitable permanent electronic storage location within the acquisition module 1010. The device ID 1046 is used by a data transmitter 1048 to modulate a load 1050 across the receiving coil 1036. The load 1050 representative of the unique device ID 1046 is reflected from the receiving coil 1036 across to the transmitting coil 1034. The current in the transmitting coil 1034 is measured across a resistance/resistor 1052 and allows a demodulator 1054 coupled thereto to determine the unique ID 1046 for the acquisition module 1010 within the holster 1018. The ID 1046 is subsequently stored in an appropriate re-writable electronic storage medium 1056 in the host module 1008 such that wireless signals from the acquisition module 1010 encoded with the ID 1046 will be compared with the stored ID 1056, recognized as being from the paired acquisition module 1010 and received for processing by the host module 1008.

In this manner, each time the acquisition module 1010 is placed within the holster 1018 of the host module 1008, the alignment of the first and second portions 1028, 1030 of the module pairing and charging system 1016 enables the battery 1044 to be charged while simultaneously pairing or re-pairing the acquisition module 1010 with the host module 1008 using the data transfer of the unique acquisition module ID 1046 from the acquisition module 1010 to the host module 1008. No further action on the part of the operator of the device 1000 is required, as the first portion 1028 of the system 1016 is constantly supplied with power by the power source for the host module 1008, e.g., a wall outlet to which the host module 1008 is connected. Further, when the acquisition module 1010 is removed from the holster 1018 for use in monitoring a patient, the wireless signals from the acquisition module 1010 will be encoded with the ID 1046. This ID 1046 will be verified with the stored value of the ID 1046 in the host module 1008 to ensure that the signals from the paired acquisition module 1010 are properly received and processed by the host module 1008 to the exclusion of any other signals that may be received by the module 1008.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A medical monitoring device comprising:
a) an acquisition module configured to receive incoming data signals from a number of sensors connected to the acquisition module concerning a physiological parameter;
b) a host module configured to receive wireless signals from the acquisition module regarding the incoming data signals received by the acquisition module; and
c) a module pairing and charging system selectively connected between the acquisition module and the host module and configured to pair the acquisition module with the host module while charging the acquisition module.

2. The device of claim 1 wherein the module pairing and charging system is an inductive module pairing and charging system.

3. The device of claim 2 wherein the module pairing and charging system includes a first portion operably connected to the host module and a second portion operably connected to the acquisition module.

4. The device of claim 3 wherein the first portion comprises:
a) a driver operably connected to a power source; and
b) a transmitting coil connected to the driver.

5. The device of claim 3 wherein the second portion comprises:
a) a unique device identifier stored in an electronic storage medium;
b) a data transmitter operably connected to the electronic storage medium; and
c) a receiving coil operably connected to the data transmitter.

6. The device of claim 6 wherein the second portion further comprises:
a) power management circuitry operably connected to the receiving coil; and
b) a battery operably connected to the power management circuitry.

7. The device of claim 3 wherein the first portion is disposed in a holster connected to the host module.

8. The device of claim 7 wherein the holster comprises:
a) a base connected to the host module and in which the first portion is located; and
b) a number of arms extending outwardly from the base to define a slot shaped complementary to the acquisition module, wherein the first portion is aligned with the second portion when the acquisition module is positioned within the slot.

9. A method for pairing an acquisition module of a medical monitoring device with a host module of the medical monitoring device, the method comprising the steps of:
a) providing a first portion of a pairing and charging system on the host module;
b) providing a second portion of the pairing and charging system on the acquisition module;
c) aligning the second portion with the first portion;
d) transmitting power from the first portion to the second portion; and
e) transmitting a unique pairing identifier from the second portion to the first portion.

10. The method of claim 9 wherein the pairing and charging system is an inductive pairing and charging system, and wherein the step of aligning the first portion with the second portion comprises placing the first portion in close proximity to the second portion.

11. The method of claim 10 wherein the first portion includes a transmitting coil the second portion includes a receiving coil, and wherein the step of aligning the first portion with the second portion comprises placing the transmitting coil in close proximity to the receiving coil.

12. The method of claim 10 wherein the first portion of the pairing an charging system is disposed in a holster disposed on the host module, and the step of aligning the first portion with the second portion comprises placing the acquisition module in the holster.

13. The method of claim 9 further comprising the steps of:
a) storing the unique identifier from the first portion in the host module after transmitting the identifier from the second portion to the first portion; and
b) comparing the stored identifier with an identifier encoded on wireless signals transmitted from the acquisition module to the host module.

14. A medical monitoring device for providing information about patient operably connected to the device, the device comprising:
a) an acquisition module including a number of sensors adapted to be attached to the patient to detect a physiological parameter of the patient and a wireless transmitter to send data signals representative of the sensed physiological parameter;
b) a host module including a wireless receiver to receive the wireless data signals from the acquisition module and a display to visually represent the data signals; and
c) an inductive module pairing and charging system selectively connected between the acquisition module and the host module; the module pairing and charging system comprising:
i. a first portion disposed on the host module and including a driver operably connected to a power source and a transmitting coil connected to the driver; and
ii. a second portion disposed on the acquisition module and including a unique device identifier stored in an electronic storage medium, a data transmitter operably connected to the electronic storage medium and a receiving coil operably connected to the data transmitter.

15. The medical monitoring device of claim 14 wherein the first portion is disposed within a holster located on the host module.

16. The medical monitoring device of claim 15 wherein the holster comprises:
a) a base connected to the host module and in which the first portion is located; and
b) a number of arms extending outwardly from the base to define a slot shaped complementary to the acquisition module, wherein the first portion is aligned with the second portion when the acquisition module is positioned within the slot.

17. The medical monitoring device of claim 14 wherein the second portion further comprises:
a) power management circuitry operably connected to the receiving coil; and
b) a battery operably connected to the power management circuitry.
